(19) United States
(12) Patent Application Publication
(10) Pub. No.: US 2010/0312315 A1
(43) Pub. Date: Dec. 9, 2010

(54) Apparatus and Method for Cooling the Brain Using a Gas

(76) Inventor: Alex Etwil, Koebenhavn N. (DK)

Correspondence Address: BRINKS HOFER GILSON & LIONE/CHICAGO/COOK, PO BOX 10395, CHICAGO, IL, 60610, US

(21) Appl. No.: 12/524,338

(22) PCT Filed: Jan. 28, 2008
(86) PCT No.: PCT/US08/01090

§ 371 Date: Nov. 16, 2009

Related U.S. Application Data
(60) Provisional application No. 60/897,745, filed on Jan. 26, 2007.

(51) Int. Cl. A61F 7/12

(52) U.S. Cl. 607/105

(57) ABSTRACT

Apparatus for cooling the brain comprises a pernasal intubation tube for insertion through one of the nostrils of a patient requiring brain cooling, an inflatable occluder provided on the tube to occlude the nasopharynx of the patient behind the soft palate of the patient and to prevent passage of liquid between the nasal cavities and the mouth of the patient; a pressure device for application to the exterior of the nose of the patient so as to apply pressure to the angular vein of the patient thereby to increase venous blood flow to the patient's brain; and a coolant gas supply for supplying coolant gas to at least one of the nasal cavities of the patient. Also described herein is a method of cooling the brain of a patient in danger of suffering brain damage comprising occluding the air passage leading from the nasal cavities to the mouth cavity of the patient behind the soft palate of the patient, applying pressure to an exterior part of the patient's nose so as to exert pressure on the angular vein of the patient thereby to increase venous blood flow to the patient's brain, and supplying a coolant gas to the nasal cavities of the patient.

### TECHNICAL FIELD

This invention relates to an apparatus and method for cooling the brain.

### BACKGROUND OF THE INVENTION

Lowering body temperature is thought to lessen the damaging effects of stroke.

There is a need in the art for an improved brain cooling apparatus which is simple and fast to use in emergency when a patient is in danger of suffering brain damage, for example, following a stroke. It is also desirable to provide a brain cooling apparatus which can be reliably and speedily put into use without significant difficulty by paramedics or nurses in a First Aid situation without requiring intervention by a physician or surgeon.

In addition, there is a need for an improved method of effecting brain cooling in a patient who has suffered a stroke or is otherwise in danger of undergoing brain damage unless speedy medical intervention is applied. In particular, there is a need for a method of effecting controlled cooling of the brain which can be put into operation simply and without the need for complex apparatus or for undertaking difficult medical procedures.

### SUMMARY OF THE INVENTION

The invention provides in one aspect an apparatus for cooling the brain comprising: a pernasal intubation tube having a proximal portion, a lumen extending from the proximal portion, a distal end for insertion through one of the nostrils of a patient requiring brain cooling, an inflatable occluder provided on a distal portion of the tube and in communication with the lumen for inflation by fluid supplied through the lumen thereby to occlude the nasopharynx of the patient behind the soft palate of the patient and to prevent passage of liquid between the nasal cavities and the mouth of the patient; a pressure device for application to the exterior of the nose of the patient so as to apply pressure to the angular vein of the patient thereby to increase venous blood flow to the patient's brain; and a coolant gas supply for supplying coolant gas to at least one of the nasal cavities of the patient.

In another aspect the invention provides a method of cooling the brain of a patient in danger of suffering brain damage comprising occluding the air passage leading from the nasal cavities to the mouth cavity of the patient behind the soft palate of the patient, applying pressure to an exterior part of the patient's nose so as to exert pressure on the angular vein of the patient thereby to increase venous blood flow to the patient's brain, and supplying a coolant gas to the nasal cavities of the patient. Such a method does not involve obstruction of the oropharynx or laryngopharynx and the patient can continue to breathe through his or her mouth with or without the aid of a respirator.

Conveniently in such a method the coolant gas is supplied through one of the patient's nostrils and discharged through the other nostril. The coolant gas can be supplied intermittently but is preferably supplied continuously.

Hence a particularly preferred method of cooling the brain of a patient suffering from a condition which could lead to brain damage comprises the steps of: providing a pernasal intubation tube having a proximal portion, a lumen extending from the proximal portion and a distal end, with an inflatable occluder provided on a distal portion in fluid communication with the lumen; introducing the distal end of the tube through one of the nostrils of the patient until the inflatable occluder lies in the nasopharynx behind the soft palate of the patient; inflating the inflatable occluder by supplying fluid through the lumen so as to occlude the nasopharynx of the patient in a region behind the soft palate of the patient, thereby to prevent passage of liquid between the nasal cavities and the mouth of the patient; applying pressure to an exterior part of the patient's nose so as to apply pressure to the angular vein of the patient thereby to increase venous blood flow to the patient's brain; supplying coolant gas to at least one of the nasal cavities of the patient; and allowing gas to exit through at least one of the patient's nostrils.

### BRIEF DESCRIPTION OF THE DRAWINGS

In the accompanying semi-diagrammatic drawings:

FIG. 1 is a vertical section through a patient's left nostril showing a pernasal intubation tube provided in accordance with the invention in position with its soft cuff inflated and with a nasal clip attached to the patient's nose;

FIG. 2 is a cross section through the right nostril of the patient of FIG. 1 with the pernasal intubation tube shown in dotted lines;

FIG. 3A is a cross section through the distal end of an intubation tube in accordance with an embodiment of the invention, showing a detailed view of its soft cuff in an inflated state;

FIG. 3B is a cross section through the distal end of an intubation tube in accordance with a further embodiment of the invention, showing a detailed view of its soft cuff in a deflated state;

FIG. 3C is a cross section through the intubation tube of FIG. 3B, displaying the soft cuff in an inflated state;

FIG. 3D is a cross section through an inflatable occluder according to a still further embodiment of the present invention, the occluder comprising two soft cuffs, which are shown in an inflated state;

FIG. 3E shows a cross section through an inflatable occluder according to a still further embodiment of the present invention, wherein the coolant gas is used to inflate the occluder;

FIG. 3F shows a cross section through an inflatable occluder according to yet another embodiment of the present invention, which is formed integrally with the intubation tube;

FIG. 3G shows a cross section through an intubation tube according to yet another embodiment of the present invention, showing the two lumens with the intubation tube;

FIG. 3H shows a cross section through an intubation according to a still further embodiment of the present invention, having a different arrangement of lumens to the embodiment of FIG. 3G;

FIG. 4 shows an inflation bulb, which is used to inflate an inflatable occluder of apparatus according to an embodiment of the present invention;

FIG. 5 shows a saline drip attachment, which is used to inflate an inflatable occluder of apparatus according to another embodiment of the present invention;

FIG. 6 shows an adapter, which may be used to connect the inflation and cooling gas sources to the intubation tube;

FIGS. 7A and 7B show the nasal clip of FIG. 1 in relaxed and expanded configurations respectively;

FIGS. 8A and 8B show a nasal clip according to a further embodiment of the present invention in relaxed and expanded configurations.

### DETAILED DESCRIPTION

Certain embodiments of the present invention provide a novel, improved form of apparatus for cooling the brain of a patient who is at risk of suffering brain damage as a result of a trauma, such as a stroke. They may provide such apparatus which can be used in a First Aid context by paramedics and other emergency service workers in situations where a person has suffered a trauma, such as a stroke, in the course of his or her normal occupations. In addition, they may provide such apparatus which can be used quickly and safely under emergency situations without risk of causing damage to the lower part of the pharynx.

Embodiments of the invention may also provide a novel, improved method of effecting brain cooling which can be speedily put into effect and which causes efficient cooling of a patient's brain following a traumatic episode, such as a stroke.

There will now be described a preferred embodiment of an apparatus for cooling the brain in accordance with the invention, which is illustrated in the accompanying drawings. This embodiment comprises a pernasal intubator comprising a tube having a proximal portion, a lumen extending from the proximal portion, a distal end for insertion through one of the nostrils of a patient requiring brain cooling, an inflatable occluder provided on a distal portion of the tube and in communication with the lumen so as to permit inflation of the inflatable occluder by fluid supplied through the lumen thereby to occlude the nasopharynx of the patient behind the soft palate of the patient and to prevent passage of fluid, i.e. liquid or gas, between the nasal cavities and the mouth of the patient. It further comprises a pressure device for application to the exterior of the nose of the patient so as to apply pressure to the angular vein of the patient thereby to increase venous blood flow to the patient's brain. In addition, it comprises a coolant gas supply for supplying coolant gas to at least one of the nasal cavities of the patient.

Preferably the lumen is provided at its proximal end with a fluid connector for connection to a source or generator of pressurised fluid. Such a fluid connector can be adapted for connection to an inflation bulb, for example.

The lumen can have a closed distal end in which case an aperture or apertures is provided in the wall of the lumen in fluid communication with the inflatable occluder whereby introduction of fluid, either liquid or a gas, through the lumen causes inflation of the inflatable occluder.

The inflatable occluder conveniently comprises a first cuff secured in fluid tight fashion to a distal portion of the tube. Alternatively the inflatable occluder may comprise a balloon secured in fluid tight fashion to a distal portion of the tube. In this case the tube may have an open distal end located within the balloon. If desired, the tube may be provided with a subsidiary inflatable cuff positioned proximally upstream from the first cuff or balloon.

Any non-toxic gas or mixture of gases can be used as coolant gas. Preferably, however, the coolant gas is oxygen. The coolant gas is preferably supplied at a temperature of from about −50° C. to about +10° C. It can be supplied, for example, at a rate of from about 1 l/min to about 100 l/min (measured at 0° C. and at 1 bar).

The pressure device conveniently comprises a nasal clip. Preferably the pressure device is adapted for securement on the tube.

In addition, there is described a method of cooling the brain of a patient in danger of suffering brain damage which comprises occluding the air passage leading from the nasal cavities to the mouth cavity of the patient behind the soft palate of the patient, applying pressure to an exterior part of the patient's nose so as to exert pressure on the angular vein of the patient thereby to increase venous blood flow to the patient's brain, and supplying a coolant gas to the nasal cavities of the patient. Such a method does not involve obstruction of the oropharynx or laryngopharynx and the patient can continue to breathe through his or her mouth with or without the aid of a respirator.

Conveniently in such a method the coolant gas is supplied through one of the patient's nostrils and discharged through the other nostril. The coolant gas can be supplied intermittently but is preferably supplied continuously.

A particularly preferred method of cooling the brain of a patient suffering from a condition which could lead to brain damage comprises the steps of:

providing a pernasal intubator comprising a tube having a proximal portion, a lumen extending from the proximal portion, a distal end, and inflatable occluder provided on a distal portion in fluid communication with the lumen;

introducing the distal end of the tube through one of the nostrils of the patient until the inflatable occluder lies in the nasopharynx behind the soft palate of the patient;

inflating the inflatable occluder by supplying fluid through the lumen so as to occlude the nasopharynx of the patient in a region behind the soft palate of the patient, thereby to prevent passage of liquid between the nasal cavities and the mouth of the patient;

applying pressure to an exterior part of the patient's nose so as to apply pressure to the angular vein of the patient thereby to increase venous blood flow to the patient's brain;

supplying coolant gas to at least one of the nasal cavities of the patient; and

allowing gas to exit through at least one of the patient's nostrils.

The tube is inserted through the patient's nostril until the inflatable occluder lies behind the patient's soft palate and then the inflatable occluder is inflated so as to block the passage of gas and liquid between the patient's nasal cavities and the patient's mouth. In this way the oropharynx and laryngopharynx of the patient are left substantially unobstructed so that the patient can continue to breathe through the mouth either unaided or with the assistance of a respirator.

Inflation of the inflatable occluder may comprise supplying air under pressure through a second lumen in the pernasal intubation tube, for example, by an inflation bulb.

Coolant gas can be supplied to the patient's nasal cavities through a second tube inserted through the patient's same nostril as the pernasal intubator or, preferably, through the patient's other nostril. Alternatively the tube of the pernasal intubator can be provided with a second lumen for supply of coolant gas terminating at an orifice in the wall of the tube of the pernasal intubator disposed at a position such that, in use, it will lie within one of the patient's nasal cavities.

Conveniently the coolant gas is supplied from a suitable supply, such as a pressurised gas cylinder, and cooled as a result of adiabatic expansion or by passage of the gas through a cooling coil immersed in a cooling bath, such as an acetone/solid CO₂ (dry ice) cooling bath or a refrigerated brine solution.

As already noted, gas is permitted to escape through one or other, or both, of the patient's nostrils. Accordingly it is necessary not to block at least one the patient's nostrils in use of the pernasal intubator.

It will normally be preferred that the temperature of the brain is monitored and that the coolant gas is introduced at a rate sufficient to maintain the temperature of the brain at between about 32° C. and about 35° C.

In the drawings the reference numerals indicate like parts throughout.

Referring to the drawings, FIG. 1 is a semi-diagrammatic cross section through part of a person's head 1, including the nose 2, right nostril 3, right nasal cavity 4, soft palate 5, lips 6 and 7, mouth 8, tongue 9, teeth 10 and 11, epiglottis 12, larynx 13, trachea 14, oesophagus 15, chin 16, nasopharynx 17, mouth cavity 18, oropharynx 19, and laryngopharynx 20.

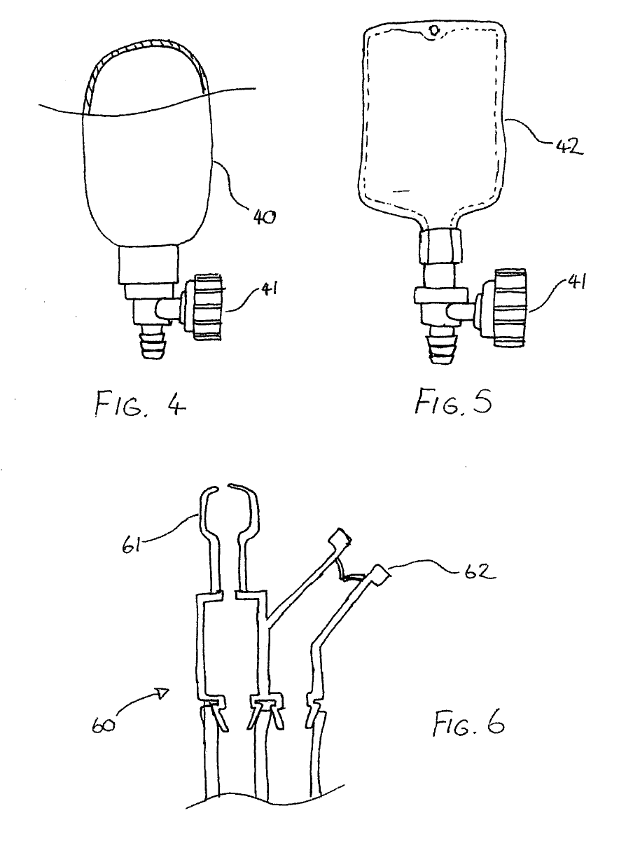

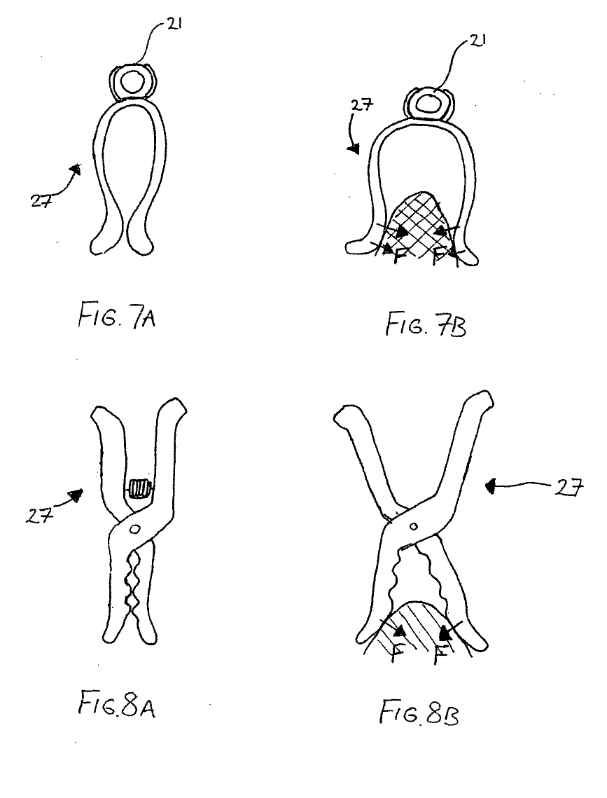

The illustrated apparatus includes a pernasal intubation tube 21 which has a lumen 22 that leads to an aperture 23 near a distal end 24 of tube 21. As illustrated the pernasal intubation tube 21 has been inserted through the patient's right nostril 3; however, it can alternatively be inserted though the patient's left nostril, if more convenient, perhaps as a result of the way that the patient is lying. Tube 21 carries an inflatable cuff 25 near its distal end 24 and aperture 23 lies within cuff 25. Tube 21 further has a proximal end 26 which is provided with an appropriate connection device (such as is shown in FIG. 6) in communication with the lumen 22 for connection to a source of an inflation fluid, such as air, oxygen, or of a liquid, such as a cold saline solution. In the embodiment shown in FIG. 4 an inflation bulb 40 similar to that used as part of a mechanical sphygmomanometer is connected to the proximal end 26 for supply of air through lumen 22 for inflating the inflatable cuff 25. The inflation bulb 40 is connected to a valve 41, which is opened or close in order to control inflation. FIG. 5 shows a cold saline dispenser 42, having a similar valve 41 to the inflation bulb 40 of FIG. 4. The saline dispenser may be a flexible polymer bag as shown in the Figure, which may be attached to a line and raised in order to generate sufficient fluid pressure. A clip 27 made of a resilient material is secured to tube 21. In use, clip 27 can be applied, as illustrated, to the external surface of the patient's nose so as to exert pressure forces F (see FIGS. 7A, 7B, 8A and 8B) on the skin and hence on the underlying angular vein of the patient thereby to increase venous blood flow to the patient's brain.

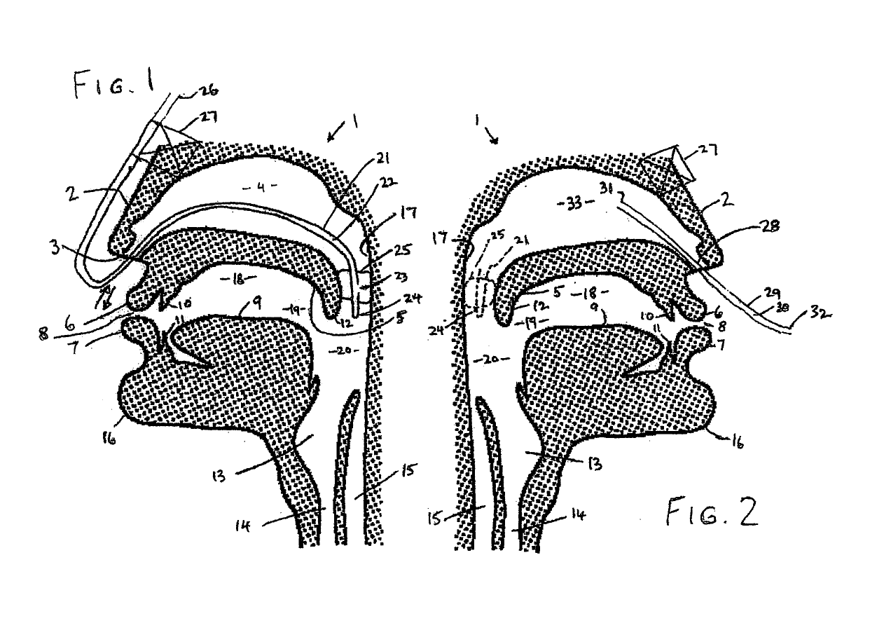

FIG. 2 shows a section through the left side of the patient's face and left nostril 28. A second tube 29 having a lumen 30 leading to an open distal end 31 and connected at its proximal end 32 to a source of coolant gas (not shown), such as cold oxygen, is inserted loosely through right nostril 28 into the right nasal cavity 33. By passing cold coolant gas through second tube 29 into the patient's nasal cavities at an appropriate rate, while pinching off the blood flow through the patient's angular vein by nasal clip 27 so as to increase the flow of venous blood to the patient's brain, the patient's brain can be cooled. Since neither pernasal intubation tube 21 nor the second tube 29 blocks either of the patient's nostrils, air and spent coolant gas, such as oxygen, can escape freely from the patient's nasal cavities.

Although the apparatus has been illustrated in the accompanying semi-diagrammatic drawings with the pernasal intubation tube 21 inserted through the patient's right nostril 3 (as shown in FIG. 1) and with the second tube 29 inserted in the patient's left nostril 28 (as shown in FIG. 2), it will be readily appreciated by the skilled reader that it does not matter which through which nostril the pernasal intubation tube 21 is inserted. Thus the pernasal intubation tube 21 can alternatively be inserted through the patient's left nostril 28 while the second tube 29 is inserted through the patient's right nostril 3.

In use of the illustrated apparatus for First Aid treatment of a patient in danger of suffering brain damage after a stroke (which is of course only one example of usages to which apparatus according to the present invention may be put), the patient will typically be lying, or made to lie, prone on his or her back or side. Having ensured that the patient is in an appropriate position, a paramedic or other medically qualified practitioner then inserts the pernasal intubation tube 21 through one of the patient's nostrils, for example, through the patient's left nostril 3, until its closed distal end 24 lies just below the patient's soft palate 5. The paramedic can then check visually through the patient's mouth 8 to confirm that the distal end 24 is in the correct position. Next the inflatable cuff 25 is inflated using, for example, an inflation bulb until inflatable cuff 25 occludes the nasopharynx 17. It will be noted that the oropharynx 19 remains substantially unobstructed so that the patient can continue to breathe through his or her mouth 8 either unaided or with the help of a respirator (not shown).

A typical inflation pressure for inflating cuff 25 is about 100 mb gauge (about 1.45 psig).

The second tube 29 is inserted in the patient's other nostril, for example, the left nostril 28.

Temperature sensors can be positioned in the patient's auditory canals or attached to the patient's neck or other areas of the head or body in order to monitor the patient's brain temperature. In addition the body temperature of the patient can be monitored with, for example, the aid of a rectal thermometer.

In a First Aid situation, once the pernasal intubation tube 21 and the second tube 29 are in place, the coolant gas supply can be turned on so as to cause coolant gas, for example, cold oxygen, to pass through second tube 29 into the patient's nasal cavities thereby cooling directly the lining of the nasal cavities 4 and 33 and also the blood in the subjacent arteries and veins.

It will be appreciated by those skilled in the art that the illustrated apparatus is simple and readily portable. It is thus well suited for use by paramedics or by persons with appropriate training in first aid, such as police officers, who are likely to be first on the scene when a person suffers a stroke in the course of his or her usual occupations. Insertion of the pernasal intubation tube 21 through one of the patient's nostrils 3 or 28 can be achieved quickly and simply without the need for any additional probe or special intubation device. Moreover it can readily be checked visually whether the distal end 24 is in the correct position behind the patient's soft palate 5 prior to inflation of cuff 25. Similarly insertion of the second tube 29 in the patient's other nostril 28 or 3 is likewise quick and simple.

In comparison with prior art techniques that involve lowering the temperature of the entire body, the various forms of apparatus and the methods described herein have the advantage that only localised lowering of temperature of the head region is effected in order to cause cooling of the brain.

If desired, an electric pump designed to produce and maintain a predetermined pressure may be used in place of an inflation bulb for the purpose of inflating cuff 25.

Any non-toxic gas or mixture of gases can be used as coolant gas for supply through tube 29. Preferably, however, the coolant gas is oxygen. The coolant gas is preferably supplied at a temperature of from about −50° C. to about +10° C. It can be supplied, for example, at a rate ranging from about 1 l/min to about 100 l/min (measured at 0° C. and at 1 bar). It can be supplied directly from a pressurised oxygen cylinder, the desired lowered temperature being achieved by virtue of adiabatic expansion of the oxygen as it emerges from the cylinder. If a lower temperature is desired, then the coolant gas can be passed through a coolant coil immersed in a cooling bath containing, for example, refrigerated brine or an acetone/solid CO₂ (dry ice) mixture. Alternatively the coolant gas can comprise air which has been bubbled through liquid oxygen.

In the course of treatment of a patient to effect brain cooling, it may be desirable that the patient's brain temperature is reduced to a temperature of not less than about +30° C., preferably not less than about +32° C., e.g. about +32° C. to about +35° C. Such reduced brain temperature can be maintained, for example, for a period of a few hours up to several few days or even months. A typical period of treatment may involve maintaining such a reduced brain temperature for from about 24 hours to about 72 hours. After receiving First Aid to reduce the brain temperature in the event of a stroke or other trauma that could lead to brain damage, the patient can undergo further suitable treatment in a hospital environment to lower his or her body temperature, in addition to reducing his or her brain temperature by the method of the invention.

The materials of construction of the apparatus of the invention can be selected from among those conventionally used in the field of medical intubation devices. For example, pernasal intubation tube 21 can be made from a pliable semi-rigid, soft plastics material such as a medical grade of polyethylene, polypropylene, polyvinylchloride, or the like. If desired, the distal end of pernasal intubation tube 21 may be coloured, e.g. blue or green, so as to increase its visibility and make it easier for a paramedic or other appropriately trained person to check that it is correctly in position before cuff 25 is inflated. The distal tip 24 may also include a radiopaque material to facilitate its visualisation in X-ray photographs.

Inflatable cuff 25 can be made from any material conventionally used for manufacture of cuffs on intubation devices, such as a medical grade of a silicone rubber.

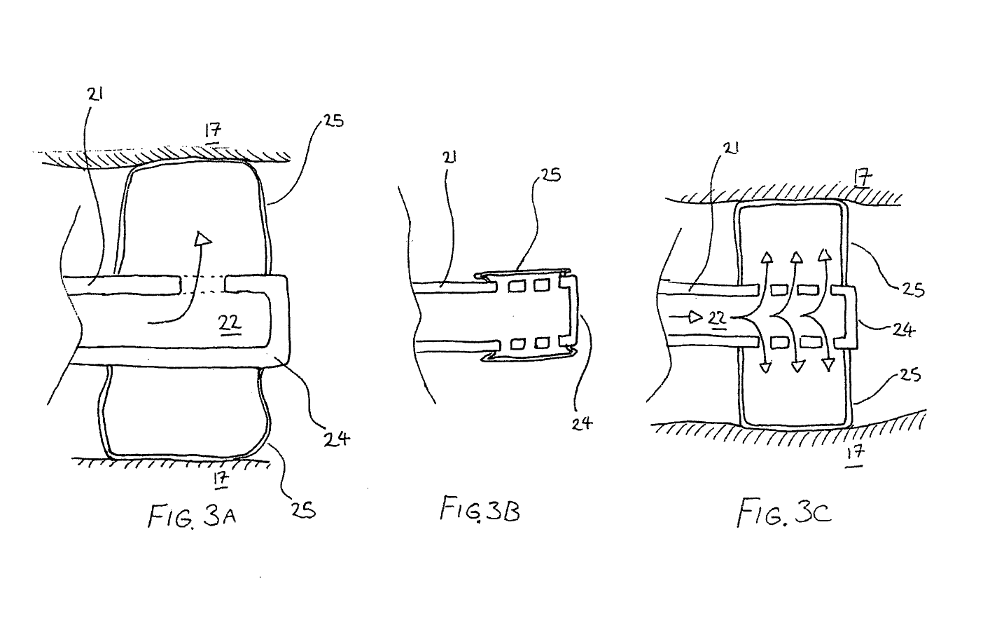

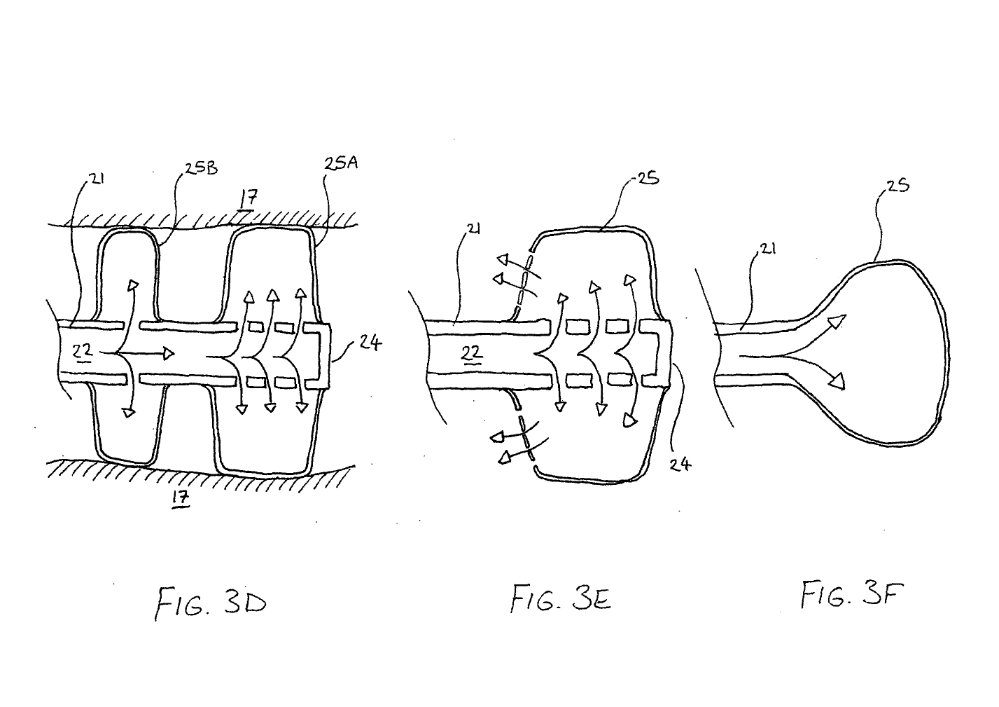

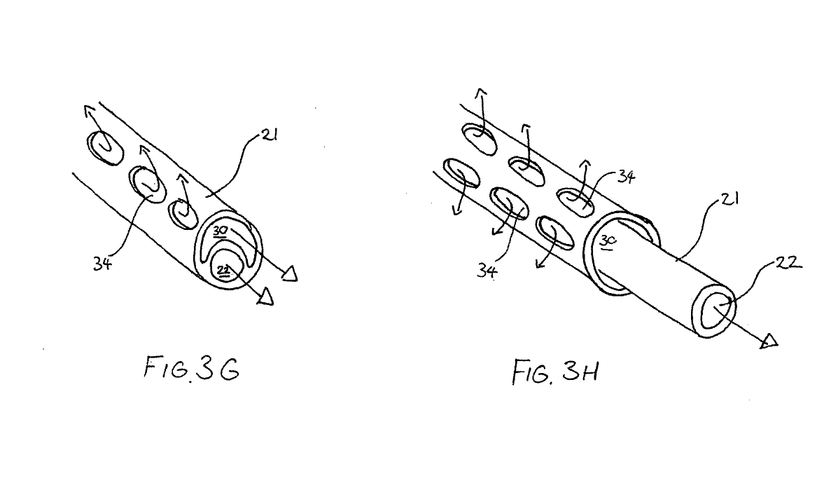

FIG. 3A shows in more detail the pernasal intubation tube 21 of FIGS. 1 and 2, having a single inflatable cuff 25 adjacent its distal end 24 which is sealed or otherwise secured in airtight fashion to the tube 21 both proximally and distally with respect to the aperture 26. FIGS. 3B and 3C show in deflated and inflated states respectively an alternative embodiment in which the tube 21 has several apertures to allow passage of the inflation fluid into the cuff 25. Alternatively, as is shown in FIG. 3F cuff 25 can be replaced by a balloon positioned at the distal end 24, being sealed or secured thereto in liquid tight fashion by a single line of sealing or the like proximally adjacent the distal end 24; in this case lumen 22 can lead to an open distal end 24 and aperture 23 is not then required. In the particular example shown in FIG. 3F, the thickness of the tube tapers so that its distal end may be inflated, thus providing an integrally formed balloon 25.

As shown in FIG. 3D, it is also envisaged that a second cuff 25B can be provided on pernasal intubation tube 21 to assist in occluding the nasopharynx, this second cuff 25B being positioned proximally with respect to cuff 25 and the lumen 22 having a further aperture or apertures for inflation of the second cuff. Alternatively the pernasal intubation tube 21 can be provided with a second lumen for inflating such a second cuff.

As described with reference to FIGS. 1 and 2, separate lines or tubes may be used to inflate the inflatable occluder and to supply cooling gas into the nasal passages.

However, it is also envisaged that these two tubes may in some constructions be combined as separate lumens of a single tube. FIGS. 3G and 3H show embodiments of the present invention having two such lumens: an inflation fluid carrying lumen 22 and a coolant carrying lumen 30. The lumens may be concentric as in FIG. 3H, but may equally be separate and parallel as in FIG. 3G. Apertures 34 are formed in the wall of the tube enclosing lumen 30 to allow egress of coolant fluid.

It is also envisaged that, in certain embodiments, the coolant gas may be used to inflate the cuff 25 of the apparatus. In such constructions a single lumen tube may be used, with apertures formed in the wall of the tube. In the particular embodiment shown in FIG. 3E, apertures are formed within the proximal surface of the inflatable cuff 25 and in the distal end of the tube 21. Thus, the inflatable cuff is inflated by the coolant gas, a portion of which is released backwards into the nasal passages to cool the brain.

As described, cuff 25 is arranged to be inflated with air under pressure. However, it is also envisaged that it can alternatively be inflated using liquid under pressure. Hence it can be arranged for connection via an inlet tap or valve to a diffusion bag filled, for example, with a cold saline solution, the appropriate pressure being generated by elevating the bag to an appropriate height above the patient's head.

A connector such as that depicted in FIG. 6 may be used for connection of the source of inflation fluid to the tube 21. This connector also includes both an adapter for the inflation fluid source 61 and a side port adapter 62 for connection of the coolant gas supply.

In place of the wishbone shaped clip of FIGS. 1, 2, 7A and 7B there may alternatively be used a hinged spring-loaded clip with two legs, opposed adjacent ends of which are urged towards each other by a spring, an exemplary construction being shown in FIGS. 8A and 8B, which is similar to that of a conventional spring-loaded laundry peg.

1. An apparatus for cooling the brain comprising:
a pernasal intubation tube having a proximal portion, a lumen extending from the proximal portion, a distal end for insertion through one of the nostrils of a patient requiring brain cooling,
an inflatable occluder provided on a distal portion of the tube and in communication with the lumen for inflation by fluid supplied through the lumen thereby to occlude the nasopharynx of the patient behind the soft palate of the patient and to prevent passage of fluid between the nasal cavities and the mouth of the patient;
a pressure device for application to the exterior of the nose of the patient so as to apply pressure to the angular vein of the patient thereby to increase venous blood flow to the patient's brain; and
a coolant gas supply for supplying coolant gas to at least one of the nasal cavities of the patient.

2. The apparatus according to claim 1 wherein the inflatable occluder comprises a first cuff secured in fluid tight fashion to a distal portion of the tube.

3. The apparatus according to claim 2 wherein the tube is provided with a subsidiary inflatable cuff positioned proximally upstream from the first cuff.

4. The apparatus according to claim 1 wherein the inflatable occluder comprises a balloon secured in fluid tight fashion to a distal portion of the tube.

5. The apparatus according to claim 4 wherein the tube is provided with a subsidiary inflatable cuff positioned proximally upstream from the balloon.

6. The apparatus according to claim 1 wherein a fluid connector is provided at the proximal end of the lumen for connection to a source or generator of pressurised fluid.

7. The apparatus according to claim 6 wherein the fluid connector is adapted for connection to an inflation bulb.

8. The apparatus according to claim 1 wherein the coolant gas supply comprises a second tube for insertion in one of the patient's nostrils.

9. The apparatus according to claim 1 wherein the pressure device comprises a clip for releasable securement on the patient's nose.

10. The apparatus according to claim 9 wherein the coolant gas supply comprises a second tube for insertion in one of the patient's nostrils and wherein the clip is adapted for securement to the second tube.

11. A method of cooling the brain of a patient in danger of suffering brain damage comprising occluding the air passage leading from the nasal cavities to the mouth cavity of the patient behind the soft palate of the patient, applying pressure to an exterior part of the patient's nose so as to exert pressure on the angular vein of the patient thereby to increase venous blood flow to the patient's brain, and supplying a coolant gas to the nasal cavities of the patient.

12. The method according to claim 11 wherein the coolant gas is supplied through one of the patient's nostrils and discharged through the other nostril.

13. The method according to claim 11 wherein a clip is applied to the patient's nose in order to apply pressure to the patient's angular vein.

14. A method of cooling the brain of a patient suffering from a condition which could lead to brain damage comprising the steps of:
providing a pernasal intubation tube having a proximal portion, a lumen extending from the proximal portion and a distal end, with an inflatable occluder provided on a distal portion in fluid communication with the lumen;
introducing the distal end of the tube through one of the nostrils of the patient until the inflatable occluder lies in the nasopharynx behind the soft palate of the patient;
inflating the inflatable occluder by supplying fluid through the lumen so as to occlude the nasopharynx of the patient in a region behind the soft palate of the patient, thereby to prevent passage of liquid between the nasal cavities and the mouth of the patient;
applying pressure to an exterior part of the patient's nose so as to apply pressure to the angular vein of the patient thereby to increase venous blood flow to the patient's brain;
supplying coolant gas to at least one of the nasal cavities of the patient; and
allowing gas to exit through at least one of the patient's nostrils.

15. The method according to claim 14 wherein the step of inflating the inflatable occluder comprises supplying air under pressure through the lumen preferably by an inflation bulb.

16. The method according to claim 14 wherein the coolant gas is supplied at a temperature of from about −50° C. to about +10° C.

17. The method according to claim 14 wherein the coolant gas is supplied at a rate of from about 1 l/min to about 100 l/min measured at 0° C. and 1 bar.

18. The method according to claim 14 wherein the temperature of the brain is monitored and wherein the coolant gas is supplied at a rate sufficient to maintain the temperature of the brain at between about 32° C. and about 35° C.

19. The method according to claim 14 wherein pressure is applied to the patient's nose by applying a clip thereto.

20. An apparatus for cooling the brain comprising:
pernasal intubation means comprising a tube having a proximal portion, a lumen extending from the proximal portion, a distal end for insertion through one of the nostrils of a patient requiring brain cooling, inflatable occluding means provided on a distal portion of the tube and in communication with the lumen so as to permit inflation of the inflatable occluding means by fluid supplied through the lumen thereby to occlude the nasopharynx of the patient behind the soft palate of the patient and to prevent passage of liquid between the nasal cavities and the mouth of the patient;
pressure applying means for application to the exterior of the nose of the patient so as to apply pressure to the angular vein of the patient thereby to increase venous blood flow to the patient's brain; and
coolant gas supply means for supplying coolant gas to at least one of the nasal cavities of the patient.
